(19) United States
(12) Patent Application Publication
(10) Pub. No.: US 2023/0167390 A1
(43) Pub. Date: Jun. 1, 2023

(54) ARRANGEMENT FOR THE CULTIVATION AND UTILIZATION OF BIOMASS AND SYSTEM OF ARRANGEMENTS FOR THE CULTIVATION AND UTILIZATION OF BIOMASS

(71) Applicant: Werner REICHERT, Regensburg (DE)

(72) Inventor: Werner REICHERT, Regensburg (DE)

(21) Appl. No.: 18/152,547

(22) Filed: Jan. 10, 2023

Related U.S. Application Data
(63) Continuation of application No. PCT/IB2021/056028, filed on Jul. 6, 2021.
Application No. PCT/IB2021/056028 is a continuation of application No. PCT/IB2021/056033, filed on Jul. 6, 2021.

(30) Foreign Application Priority Data
Jul. 10, 2020 (DE) 10 2020 118 208.2
Jul. 10, 2020 (DE) 10 2020 118 210.4

(51) Int. Cl. C12M 1/107; C12M 1/36

(52) U.S. Cl. CPC C12M 21/04; C12M 41/48

(57) ABSTRACT

An arrangement (1) for the cultivation of plants and for the utilization of biomass waste and a system (1000) of at least one arrangement (1) for the cultivation and utilization of biomass are disclosed. The arrangement (1) comprises a modular greenhouse (100) and a modular, two-stage biogas plant (200). The system (1000) has at least one arrangement (1) wherein a control and monitoring unit (101) of the modular greenhouse (100) and a further a control and monitoring unit (201) of the two-stage biogas plant (200) are communicatively connected to a central control and monitoring unit (55).

### CROSS-REFERENCE TO RELATED APPLICATIONS

The present application is filed under 35 U.S.C. §§ 111(a) and 365(c) as a continuation of International Patent Application No. PCT/M2021/056028, filed on Jul. 6, 2021, which application claims priority from German Patent Application No. DE 10 2020 118 208.2, filed on Jul. 10, 2020, and as a continuation of International Patent Application No. PCT/M2021/056033, filed on Jul. 6, 2021, which application claims priority from German Patent Application No. DE 10 2020 118 210.4, filed on Jul. 10, 2020 which applications are incorporated herein by reference in their entireties.

### FIELD

The invention relates to an arrangement for the cultivation and utilization of biomass. Additionally, the invention relates to a system of at least one arrangement for the cultivation ant utilization of biomass

### BACKGROUND

German utility model DE 94 09 728 U1 discloses a greenhouse. The greenhouse is modularly constructed from several segments. Each segment consists of at least two side panes, as well as at least two roof panes, which form a gable roof. Pin-shaped connecting elements serve to establish a stable connection with a second profile rail, forming the roof ridge, of the adjacent segment.

Austrian patent application AT 511 169 A1 discloses a modular building. In this context, the modular building comprises two or more building modules, each of which has a load-bearing skeleton comprising vertical supports arranged on the vertical edges of a cuboid and floor and ceiling beams arranged on the horizontal edges of the cuboid. The load-bearing skeletons of the adjacent building modules are connected to each other by bolted connections.

German utility model specification DE 20 2007 005 638 U1 discloses a modular greenhouse. The modular greenhouse comprises a framework-like frame. For example, translucent walls can be held in the frame. A pavilion section consists of at least three, in particular four, horizontal subsections forming approximately a semicircle. The inclined roof elements are isosceles triangles whose vertices converge.

German patent application DE 10 2008 015 609 A1 discloses a biogas plant and a process for producing biogas. Disclosed herein is a method for producing biogas, in particular methane gas, in a multi-stage process. The multi-stage process includes a hydrolysis process and a methane formation process. The hydrolysis process is spatially separated from the methane formation process. The biogas plant itself has at least two hydrolysis tanks and a fermentation tank or fermenter for a methane formation process. The at least two hydrolysis tanks are spatially separated from the downstream fermenter. A disadvantage of this biogas plant is that it is not modular, not mobile and not compact in design. In addition, this biogas plant does not provide for remote maintenance or remote control. It is not possible to move the plant to another location or to set it up differently on site.

German utility model DE 20 2013 101 554 U1 discloses a container arrangement of a biogas plant. The container has a bottom and a circumferential wall. The wall of the container is outwardly supported against at least one container. The at least one container contains a technical device required for operating the biogas plant.

German patent application DE 199 58 142 A1 discloses a modular biogas plant. The transportable, modular biogas plant comprises a fermenter and an energy part, both of which are separate construction elements. These components are housed in standard transport containers and standard transport container frames, respectively. The fermenter has a rigid shell. The biogas plant described here is a single-stage and non-thermophilic process.

German patent application DE 10 2014 016 801 A1 discloses a process for demand-oriented generation of electrical power and demand-oriented provision of thermal energy by means of at least one biogas plant. The biogas produced is converted into electricity in at least one combined heat and power plant, generating thermal energy.

German patent application DE 10 2009 028 474 A1 discloses a photobioreactor with at least one photosynthesis unit, a piping system with valves and at least one system pump and a monitoring and control device. At least the photobioreactor is arranged in a container, which can be transported as a cargo unit without additional protective and stabilizing measures or protective elements and can be stacked with other containers. The photobioreactor with a harvesting device and a cleaning and disinfection device form an integral unit and are arranged together in one container.

German utility model specification DE 200 16 591 U1 describes a plant for the production of biogas, with a preliminary pit for the homogenization and intermediate storage of biogenic waste, a tubular fermenter, a gas storage tank and an energy center.

International patent application WO 2005/101 525 A2 covers combining one or more photovoltaic cells. These cells are suitable for containing one or more photosynthetic organisms. The photovoltaic cells may be configured to be at least partially transparent to light of at least one wavelength capable of driving photosynthesis, such as at least one wavelength between about 400 nm and about 700 nm.

German patent application DE 10 2012 214 493 A1 discloses a photobioreactor for the cultivation of phototrophic organisms. A transparent pipe system is provided for the flow of a culture suspension, in particular algae substrate. The transparent pipe system is designed in the form of tiers to enable particularly efficient cultivation over several tiers.

European patent EP 1 100 867 B1 describes an integrated fermenter and a greenhouse. The fermenter is located in the greenhouse. The digester is an anaerobic digester for organic waste material.

International patent application WO 2012/100 093 A2 discloses systems, components and processes directed to the production of energy and feedstock from biomass in a biorefinery system. The systems, components and processes can be used alone or in combination as part of an integrated biorefinery system.

International patent application WO 2016/180068 A1 discloses a modular lightweight building, typically comprising a plurality of modules. The modular building may be composed of modules of the same construction type or of modules of different construction types. In this context, it is convenient that the modules of the second construction type are selected such that they can accommodate the modules of the first construction type for transportation purposes.

International patent application WO 2014/126474 A1 discloses a building that can be placed on a surface. The building comprises an inner supporting structure and an outer shell.

International patent application WO 2008/058950 A1 discloses a cover element for greenhouses or the like. The cover element comprises: at least a first substantially transparent layer having alternating stripes with at least one optical means for deflecting and concentrating light. Further, at least a second partially opaque layer comprising alternating strips of optically neutral and substantially transparent strips alternated with opaque strips and consisting of at least one photovoltaic element.

None of the above documents describes or mentions the combination or interconnection of a greenhouse (facility for the production or cultivation of plants; essentially plants for food) and a biogas plant (facility for the utilization of biomass waste of any kind).

### SUMMARY

The object of the invention is therefore to provide an arrangement at least for the cultivation of plants and for the utilization of biomass waste, which is essentially energy self-sufficient and produces biomass in the form of plants and at the same time utilizes biomass waste and obtains an energy carrier.

This object is achieved by an arrangement for the cultivation of plants and for the utilization of biomass waste, which is defined by a modular greenhouse and a modular two-stage biogas plant. The modular greenhouse is composed of a plurality of modules and a control and monitoring unit is associated with the modular greenhouse. The modular two-stage biogas plant is defined by several modules, wherein the modules of the modular two-stage biogas plant are partly formed as tanks, comprising at least two hydrolysis tanks and at least one fermentation tank. A further control and monitoring unit is associated with the two-stage biogas plant. A communication link is provided between the control and monitoring unit of the modular greenhouse and the further control and monitoring unit of the modular, two-stage biogas plant, so that energy from the biogas in the form of light and/or heat is used on request for the cultivation of plants in the modular greenhouse.

The advantage of the arrangement according to the invention is that it creates an essentially energy-neutral greenhouse for growing plants, which can be used in all climatic zones of the world, thus enabling multiple harvests. In addition, the two-stage biogas plant can be used to process not only biomass waste from the greenhouse itself but also from many different sources. The electrical energy generated by the greenhouse can be fed into an energy storage system for later on request use in the greenhouse.

It is a further object of the invention to provide a system of at least one arrangement for the cultivation ant utilization of biomass waste, which is essentially energy self-sufficient and produces biomass in the form of plants and an enables a central control of the at least one arrangement.

The above object is achieved by a system of at least one arrangement for the cultivation and utilization of biomass. The at least one arrangement is defined by a modular greenhouse and a modular two-stage biogas plant composed of a plurality of modules and a control and monitoring unit associated with the greenhouse. A plurality of modules defines the modular greenhouse and a control and monitoring unit is associated with the greenhouse. A further control and monitoring unit is associated with the modular two-stage biogas plant, wherein the modular two-stage biogas plant consists of several modules and the modules of the modular two-stage biogas plant are partly formed as tanks, having at least two hydrolysis tanks and at least one fermentation tank. A communication link is provided between the control and monitoring unit of the arrangement of the modular greenhouse and the further control and monitoring unit of the modular, two-stage biogas plant. A central control and monitoring unit is communicatively connected to a control and data acquisition unit of the modular greenhouse and to a further control and monitoring unit of the modular, two-stage biogas plant.

The advantage of the system of at least one arrangement for the cultivation and utilization of biomass is that the central control and monitoring unit enables remote maintenance and trend analyses of at least one arrangement. A user of the at least one arrangement can be alerted to possible errors or failures at an early stage, which reduces the downtime of the at least one arrangement and increase efficiency.

According to an embodiment of the invention the modules of the two-stage biogas plant can be designed so that they all have the same size and are stackable.

The advantage that all modules of the biogas plant have the same size and correspond to a standard container is that transport and logistics are greatly facilitated. Another facilitation is when the individual components of the modules of the greenhouse are delivered prefabricated in a container that has the same size as the modules of the biogas plant.

According to an embodiment of the invention each module of the greenhouse, has a roof with at least one transparent roof surface which supports at least one photovoltaic module which is at least partially transparent or adjustable with respect to transparency. An energy storage device is provided for the at least one photovoltaic module of the greenhouse. The energy storage device can be a battery. According to an additional embodiment the energy storage device is a power-to-gas system, with which hydrogen or methane can be generated and stored from the electricity generated by the photovoltaic modules. A further embodiment is that the energy storage device is a power-to-liquid system, with which methanol can be generated and stored from the electricity generated by the photovoltaic modules.

In the smallest expansion stage, the two-stage biogas plant comprises at least two hydrolysis tanks and at least one fermentation tank. In the hydrolysis tank, the biomass is exposed to a temperature range of 40 to 65° C. and a ph value during hydrolysis of 2 to 9. Furthermore, at least one gas storage tank is provided for the biogas produced in the modular biogas plant. Initially, the hydrolysis tanks are filled with biomass in batches. A temperature is set in the hydrolysis tanks according to a first temperature range. During hydrolysis, the pH value in the hydrolysis tanks is within a predefined pH range. Batch filling of the hydrolysis tanks means that one tank is filled almost completely depending on a predefined time interval and hydrolysis takes place in the other tank.

In the at least one fermenter tank (digester tank), the production of biogas takes place from the biomass transferred from the hydrolysis tank to the fermenter tank. The production of biogas takes place in a second temperature range, such as from 35 to 60° C. and at a second pH range, such as from 6.5 to 8.5. The fermentation in the fermentation tanks is an acetic acid formation (an acidification) and divided into a methanization.

The advantage of the two-stage biogas plant is that the two-stage biogas plant can digest all types of biomass.

According to an embodiment of the invention, the system has an associated firewall which is provided for each arrangement. The control data acquisition unit of each modular greenhouse and the further local and the control and monitoring unit of each modular two-stage biogas plant is connected via the firewall and a cloud to the central control and monitoring unit.

According to an invention the system has plural arrangements, each having a single modular greenhouse and a two-stage modular biogas plant. Each arrangement can be designed in such a way that it can communicate with a central control and monitoring unit. The communication of the individual arrangements with the central control and monitoring unit can take place, for example, via the cloud. The data and parameters, of the individual arrangements (greenhouse and biogas plant) are evaluated with the central control and monitoring unit. Each of the arrangements includes a local control and data acquisition unit of the modular greenhouse and a control and monitoring unit of each modular, two-stage biogas plant. Each arrangement communicates with a central control and monitoring unit via local control and data acquisition unit of the modular greenhouse and the further control and monitoring unit of each modular, two-stage biogas plant. Accordingly, the user of the at least one arrangement can receive an advice and/or instructions from the central control and monitoring unit sent to a user interface which can be e.g. a cell phone, a tablet or a laptop. The system enables as well a condition-based maintenance (CBM) to be implemented. The CBM functions of remote maintenance as well as trend analyses allow the user of the arrangement to be alerted to possible errors or failures at an early stage.

A further embodiment of the invention is an arrangement for the cultivation of plants and for the utilization of biomass waste. The arrangement has a modular greenhouse composed of a plurality of modules. Each of the modules is defined by a first frame element, a second frame element, a third frame element, a fourth frame element and a roof. Each module has at least two roof structures which hold at least one transparent roof surface. At least one photovoltaic module is mounted on the roof surface, wherein the photovoltaic module is designed to be partially transparent or adjustable in terms of transparency. A control and monitoring unit associated with the modular greenhouse. The arrangement has as well a modular two-stage biogas plant which has several modules, wherein the modules of the modular two-stage biogas plant are partly formed as tanks, configured by at least two hydrolysis tanks and at least one fermentation tank. A further control and monitoring unit is associated with the two-stage biogas plant. A communication link between the control and monitoring unit of the modular greenhouse and the further control and monitoring unit of the modular two-stage biogas plant are provided, so that energy from the biogas in the form of light and/or heat is used on request for the cultivation of at least plants in the modular greenhouse. A firewall is assigned to each arrangement, wherein the control and data acquisition unit of the modular greenhouse and the further local and the control and monitoring unit of the two-stage biogas plant communicate via the firewall with the outside world.

These and other objects, features, and advantages of the present disclosure will become readily apparent upon a review of the following detailed description of the disclosure, in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1 is a schematic view of a possible embodiment of the biomass cultivation and utilization arrangement;

FIG. 2 is a schematic view of another embodiment of the biomass cultivation and utilization arrangement;

FIG. 3 shows a schematic top view of an energy-efficient greenhouse connected to an energy storage system;

FIG. 4 shows a schematic plan view of the floor plan of a single module that can be used to construct an energy-efficient modular greenhouse;

FIG. 5 shows a schematic perspective view of a module that can be used for the construction of a modular greenhouse;

FIG. 6 shows a schematic view of a possible embodiment of the structure of a roof for the individual modules;

FIG. 7 shows a top view of a possible embodiment of the photovoltaic module, which is provided with a thin-film solar cell that is partially transparent;

FIG. 8 is a sectional view of the photovoltaic module along the sectional line A-A shown in FIG. 7;

FIG. 9 is a top view of a further possible embodiment of the photovoltaic module with partially transparent thin-film solar cells;

FIG. 10 is a top view of a still further possible embodiment of the photovoltaic module, which is provided with movable lamellae;

FIG. 11 shows a sectional view of the embodiment shown in FIG. 10 along the line of intersection B-B;

FIG. 12 shows a possible embodiment of the lamellae used;

FIG. 13 shows another possible embodiment of the lamellae used;

FIG. 14 shows yet another possible embodiment of the lamellae used;

FIG. 15 shows a schematic side view of a modular greenhouse constructed from several modules;

FIG. 16 shows a schematic top view of a modular greenhouse constructed from several modules arranged in matrix form;

FIG. 17 shows a perspective schematic view of a container for transporting module or modules disassembled into individual parts;

FIGS. 18A to 18D show a preferred embodiment of the dimensioning of the individual components of a module so that a container can be optimally filled;

FIG. 19 is a top view of a possible design of a modular biogas plant;

FIG. 20 is a side view of an embodiment of a module designed as a tank and used in the modular biogas plant according to the invention;

FIG. 21 is a front view of the module according to FIG. 20;

FIG. 22 is a schematic view of the arrangement of the various modules of an embodiment of the modular biogas plant according to the invention;

FIG. 23 is a schematic view of the modular and energy efficient greenhouse; and

FIG. 24 is a schematic view of an embodiment of the system according to the invention, how the individual modular biogas plants and the modular greenhouses communicate with a central control and monitoring system.

### DETAILED DESCRIPTION

In the figures, identical reference signs are used for identical or similarly acting elements of the invention. Furthermore, for the sake of clarity, only reference signs that are necessary for the description of the respective figure are shown in the individual figures. The embodiments shown merely represent examples of how the modular greenhouse and the modular biogas plant may be configured. The illustrated embodiments are not to be understood as a limitation of the invention. The size ratios of the individual elements to each other in the figures do not always correspond to the real size ratios, since some shapes are simplified and other shapes are shown enlarged in relation to other elements for better illustration.

Furthermore, it is understood that this disclosure is not limited to the particular methodology, materials and modifications described and as such may, of course, vary. It is also understood that the terminology used herein is for the purpose of describing particular aspects only and is not intended to limit the scope of the claims.

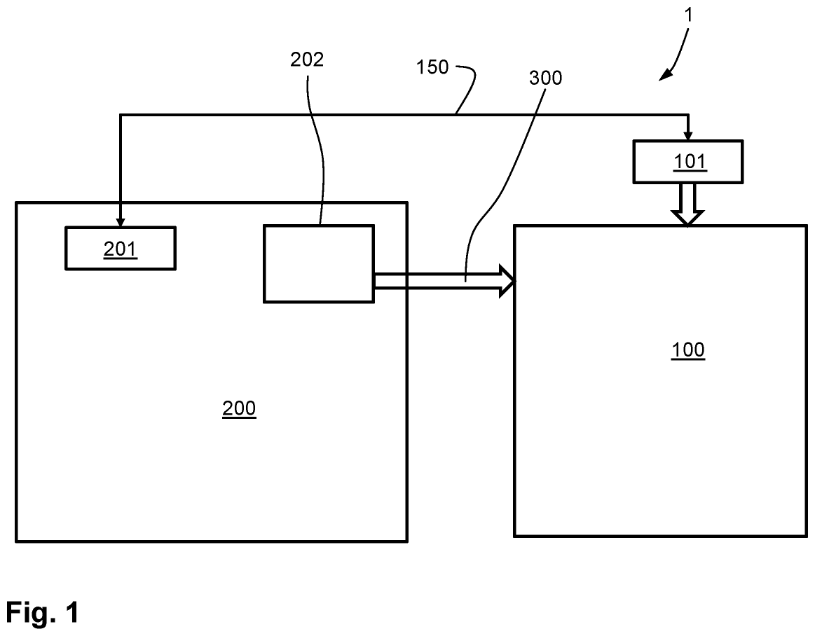

FIG. 1 shows a schematic view of a possible embodiment of arrangement 1 for biomass production and utilization. The arrangement 1 comprises a modular greenhouse 100 and a modular, two-stage biogas plant 200. The modular greenhouse 100 is used for biomass production and has a control and monitoring unit 101. The biomass are plants which are gown mainly for food purposes. The control and monitoring unit 10₁, is used for determining measured variables in or on the greenhouse 100 and for adjusting elements of the greenhouse to set a predefined value.

The modular, two-stage biogas plant 200 has further a control and monitoring unit 201. The control and monitoring unit 101 of the greenhouse 100 is connected to the further control and monitoring unit 201 of the modular, two-stage biogas plant 200 via a communication link 150. The communication link 150 may be implemented in a wired or wireless manner. The modular, two-stage biogas plant 200 has a gas storage unit 202 for the biogas produced by the modular, two-stage biogas plant 200. In the event that the greenhouse 100 requires energy 300 in the form of light or heat, the energy can be generated with the biogas. The request for energy is based on the measurements of the control and monitoring unit 101 of the greenhouse 100.

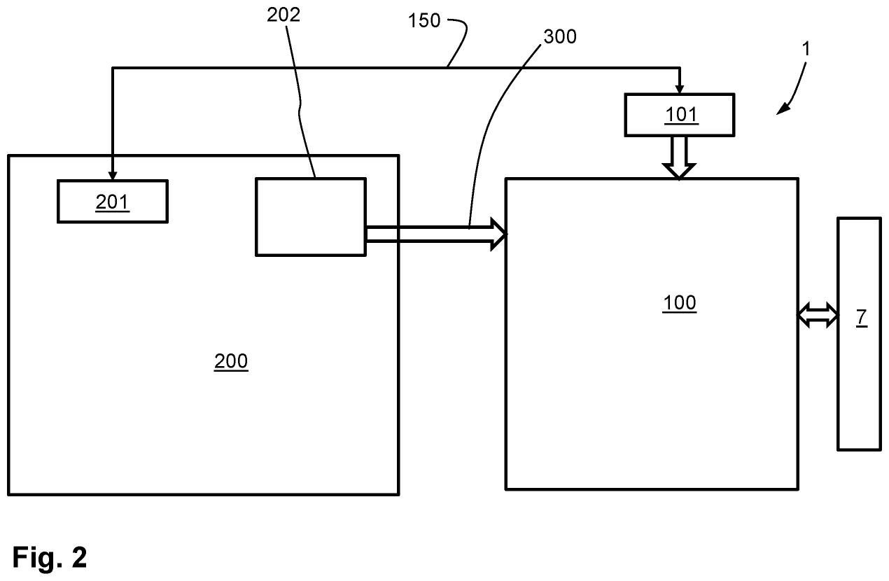

FIG. 2 shows a schematic view of a further embodiment of the arrangement 1 for cultivation (growing of plants) and utilization of biomass. The only difference to the embodiment according to FIG. 1 is that an energy storage unit 7 is associated with the greenhouse 100.

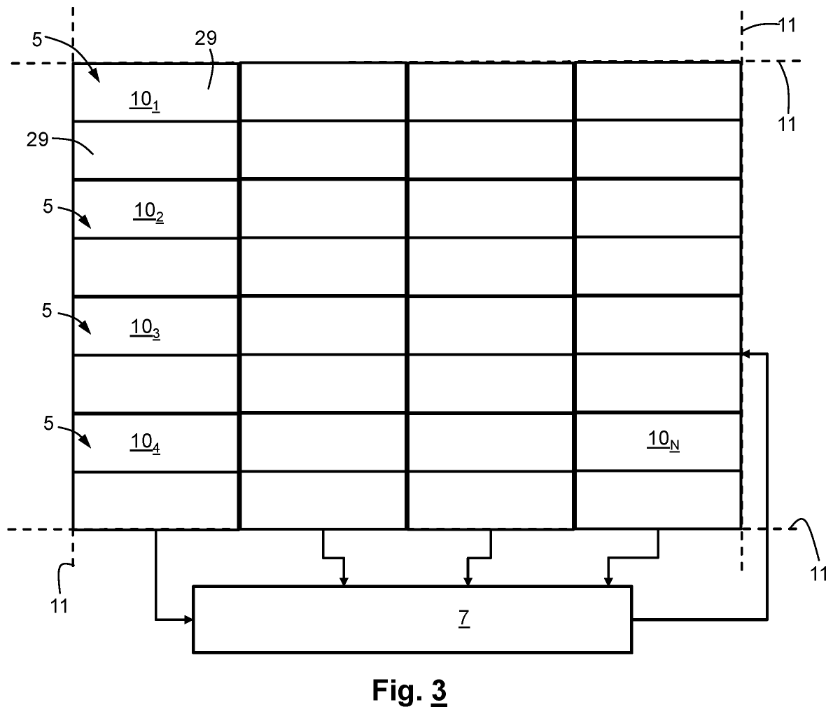

FIG. 3 shows a schematic top view of an energy-efficient greenhouse 100 associated with an energy storage unit 7 (see also FIG. 2). Here it can be clearly seen that the energy-efficient greenhouse 100 is constructed from a plurality of individual modules 10₁, 10₂, . . . , 10_{N} arranged in the form of a matrix. The transparent roof surfaces 5 of the individual modules 10₁, 10₂, . . . , 10_{N} support the photovoltaic modules 29. Those modules which are arranged on the outer sides 11 of the energy-efficient greenhouse 100 have transparent side surfaces 6 (see FIG. 5). The transparent side surfaces 6 may also have photovoltaic modules 29 (not shown here). Depending on the climatic region in which the greenhouse 100 is located, the transparent side surfaces 6 may be omitted. The photovoltaic modules 25 of the energy efficient greenhouse 100 are electrically connected to the energy storage device 7. The energy storage 7 can be used to supply the energy efficient greenhouse 100 with energy, such as light energy and/or heating energy.

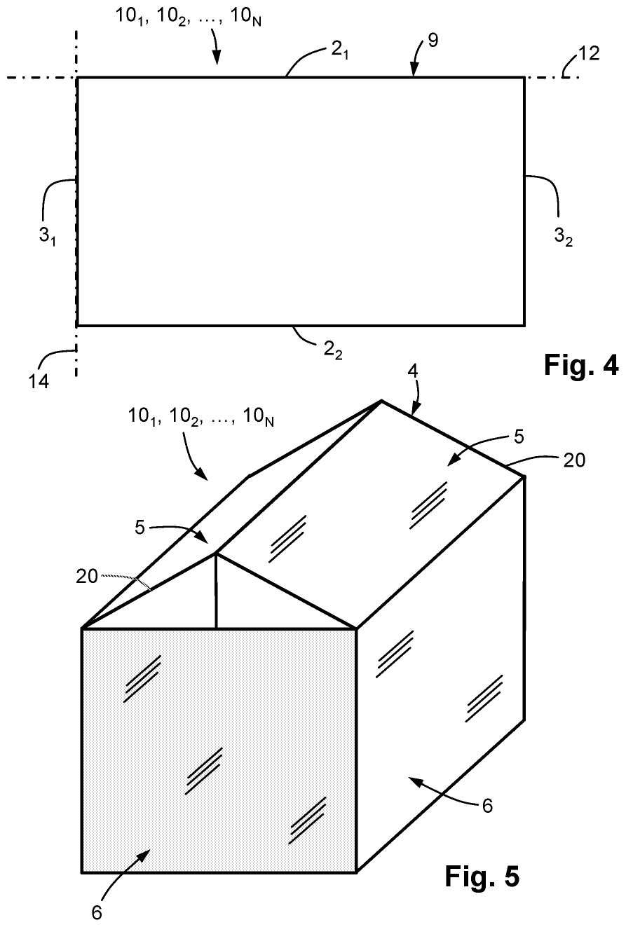

FIG. 4 shows a schematic perspective view of a floor plan 9 of an embodiment of a single module 10₁, 10₂, . . . , 10_{N} that can be used for the construction of an energy efficient modular greenhouse 1. The single module 10₁, 10₂, . . . , 10_{N} defines two longitudinal sides 12 at which, a first frame element 2₁ and a second frame element 2₂ are arranged parallel to each other. The single module 10₁, 10₂, . . . , 10_{N} also defines two transverse sides 14 at which, a third frame element 3₁ and a fourth frame element 3₂ are arranged parallel to each other. When the first frame element 2₁ and the second frame element 2₂ are arranged at a right angle to the third frame element 3₁ and the fourth frame element 3₂, respectively, the floor plan 9 has the shape of a rectangle.

FIG. 5 shows a schematic perspective view of a module 10₁, 10₂, . . . , 10_{N} which can be used for the construction of an energy efficient modular greenhouse 100. At least two roof structures 20 are provided for forming a roof 4 of each module 10₁, 10₂, . . . , 10_{N}. The at least two roof structures 20 of each module 10₁, 10₂, . . . , 10_{N} carry transparent covers 30 (see e.g. FIG. 8), which form the transparent roof surfaces 5. The transparent roof surfaces 5 are associated with the plurality of photovoltaic modules 25 (see, e.g., FIGS. 7-11).

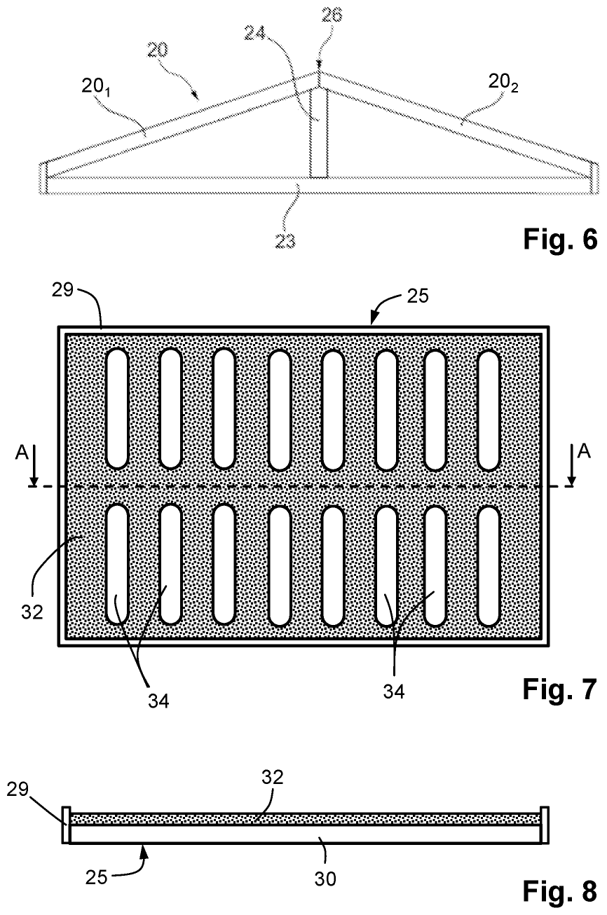

FIG. 6 shows a front view of a possible embodiment for a roof structure 20 that can be used to form the roof 4. In the embodiment shown here, the roof structure 20 has the shape of an obtuse-angled isosceles triangle. It should be noted here that the shape of the triangle of the roof structure 20 should not be taken as a limitation of the invention. It is self-evident to one skilled in the art that the triangle of the roof structure 20 may take any shape up to and including a right-angled triangle. The roof structure 20 includes a first leg 20₁ and a second leg 20₂. The first leg 20₁ and the second leg 20₂ are connected to each other by a base 23 of the roof structure 20 shown herein.

A support 24, if required for structural reasons, connects a top 26 of the support structure 20 to the base 23. At least two roof structures 20 connecting the first frame member 21 and the second frame member 22 are required for mounting the cover 25.

Although the embodiment of the roof structure 20 shown in FIG. 6 is an isosceles triangle, this should not be taken as a limitation of the invention. It is obvious to one skilled in the art that other roof structures 20 are possible for the modules 10₁, 10₂, . . . , 10_{N} of the energy efficient modular greenhouse 1.

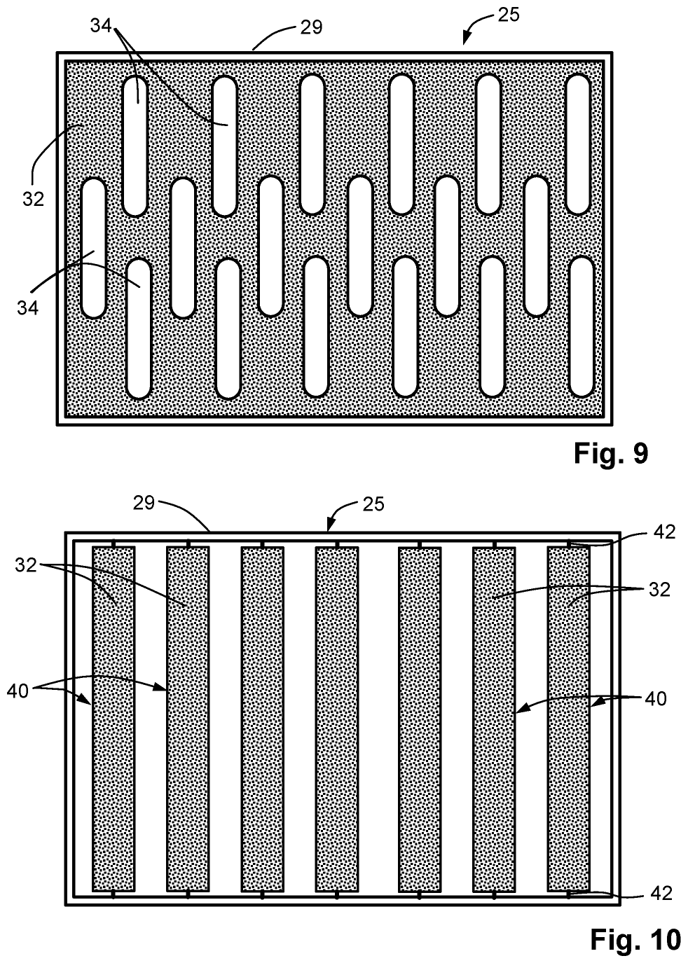

FIG. 7 shows a top view of a possible embodiment of an at least partially transparent photovoltaic module 25, which is supported or carried by the roof structure 20 (see FIG. 6). The photovoltaic module 25 is surrounded by a frame 29. As can be seen from the illustration of FIG. 8, which is a view along section line A-A, at least one thin-film solar cell 32 is applied to a transparent cover 30, such as a glass pane or a plastic pane. The at least one thin-film solar cell 32 is provided with a plurality of recesses 34 (see FIG. 7). By means of the recesses 34, the partially transparent property of the photovoltaic module 25 can be achieved. FIG. 9 shows another embodiment of the arrangement of the recesses 34 in the thin-film solar cell 32. As desired, the number of recesses 34 can be designed in such a way that a transparency of 20%, 30% or 40% is set for a photovoltaic module 25. The aforementioned figures regarding transparency are not to be understood as a limitation of the invention.

FIG. 10 shows a top view of yet another possible embodiment of the photovoltaic module 25, which is provided with movable lamellas 40. The lamellas 40 are movably arranged in the frame 29 of the photovoltaic module 25. The lamellas 40 are pivotable about axes 42. The pivoting of the lamellas 40 around the axes 42 can be done by means of a control (not shown), in such a way that the lamellas 40 always take the optimal position to the sun, so that the energy yield is optimized. Likewise, the lamellas 40 can be controlled in such a way that a defined shading for the interior of the greenhouse 100 can be set.

FIG. 11 shows a sectional view of the embodiment shown in FIG. 10 along section line B-B. The pivotable lamellas 40 are arranged above the transparent cover 25. The pivoting lamellas 40 are arranged in the frame 29 and, depending on the pivoting position, can provide greater light transmission or shading.

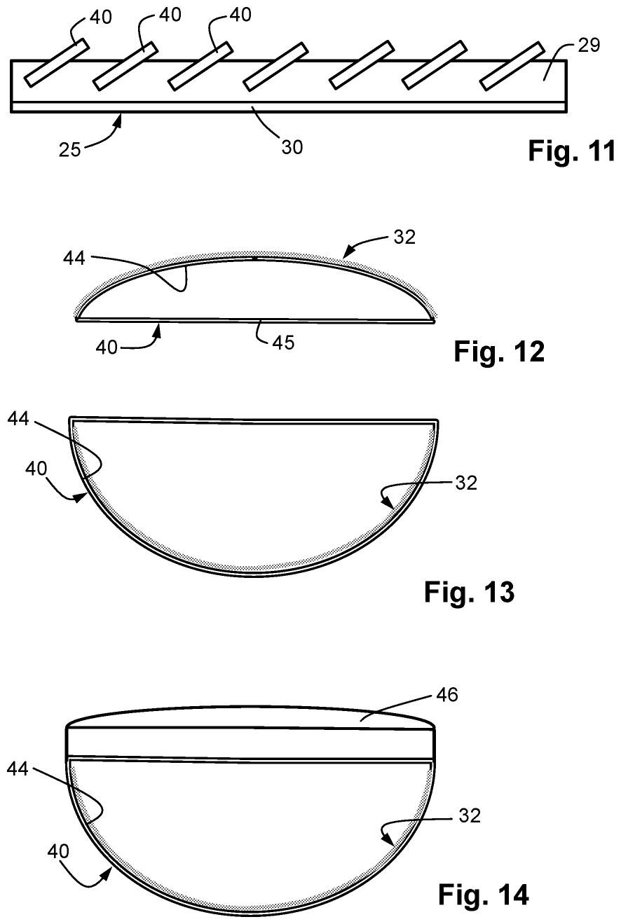

FIG. 12 shows a cross-sectional view of a possible embodiment of the lamellae 40 used. A surface 44 of the lamella 40 has a convex curvature with a substantially rectangular base 45. The thin-film solar cell 32 is applied to the surface 44.

FIG. 13 shows another possible embodiment of the lamella 40 used. The lamella 40 has a concave shape. The thin-film solar cell 32 is applied to the concave curvature (surface 44).

FIG. 14 shows yet another possible embodiment of the lamellae 40 used. Each lamella 40 is concavely shaped analogous to the lamella 40 of FIG. 13. The thin-film solar cell 32 is applied in the concave curvature (surface 44). Opposite the thin film solar cell 32, an optical element 46 is provided to concentrate the incident light onto the thin film solar cell 32.

The embodiments of the lamellae 40 shown in FIGS. 12 to 14 should not be construed as limiting the invention. The illustrated lamella 40 merely represent possible embodiments. Similarly, lamellae 40 of different types may be combined in a frame 29 of the cover 25. Likewise, in particular applications, of the cover 25 with the lamellae 40, the transparent cover 30 may be omitted.

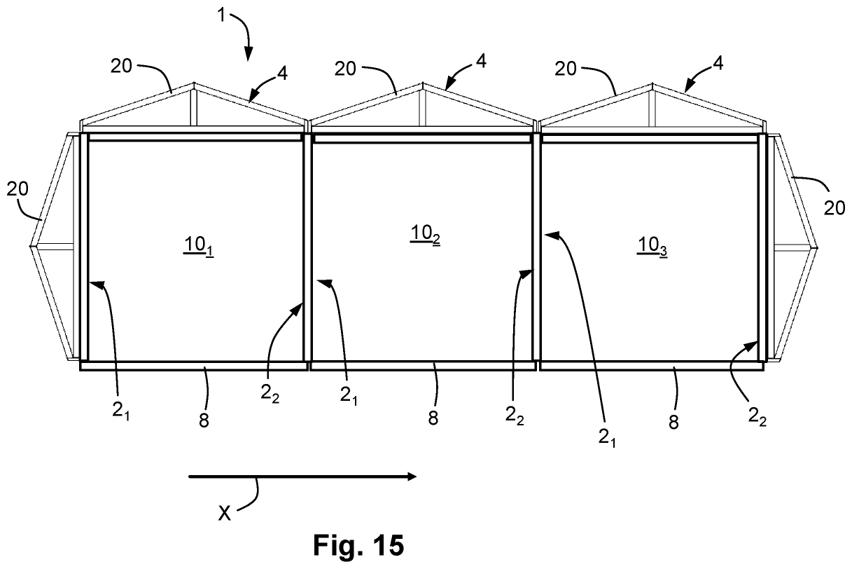

FIG. 15 shows a schematic side view of an embodiment of the greenhouse 1 in which three modules 10₁, 10₂ and 10₃ are connected to each other in the direction X shown here. The module 101, which is placed at one end of the modular greenhouse 1, carries the at least two roof structures 20 for the roof 4. At the first frame element 2₁, this module 10₁ also carries the at least two roof structures 20. The module 10₃ at the opposite end of the greenhouse 1 carries at least two roof structures 20 and at the second frame element 2₂ of the module 103 at least two roof structures 20 are also provided. The modules 10₁, 10₂, . . . , 10_{N} of the greenhouse 1 are interconnected with adjacent modules 10₁, 10₂, . . . , 10_{N}. Each of the modules 10₁, 10₂, . . . , 10_{N} may further be provided with a floor element 8.

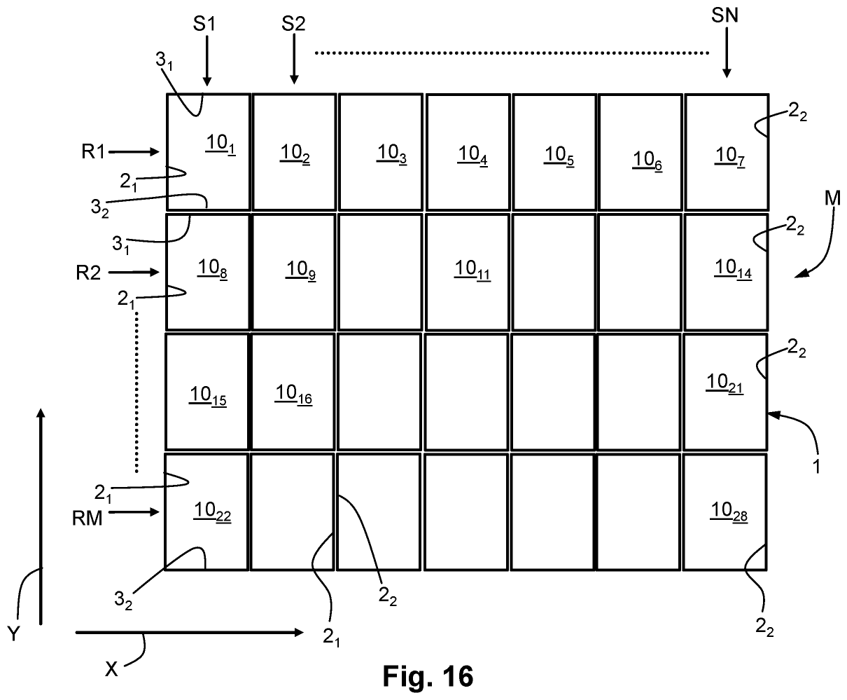

FIG. 16 shows a top view of a modular greenhouse 1, in which the individual modules 10₁, 10₂, . . . , 10_{N} are arranged in a matrix M. The rows R1, R2, . . . , RN extend in the X-direction and the columns S1, S2, . . . SN, extend in the Y-direction. Each of the modules 10₁, 10₂, . . . , 10_{N} of the greenhouse 1 provided with a roof 4 (see FIG. 5). Prior to assembly to form the modular greenhouse 1, each of the modules 10₁, 10₂, . . . , 10_{N} may be provided with a floor element 8 (see FIG. 15). The roof 4 comprises the at least two roof structures 20. The modules 10₁, 10₈, 10₁₅ and 10₂₂ of the first column S1 and the modules 10₇, 10₁₄, 10₂₁ and 10₂₈ of the last column SN are each provided with at least two roof structures 20 (not shown) on the first frame element 21 and the second frame element 22, respectively. To form the modular greenhouse 1, the modules in columns S1, S2, . . . , SN are connected to a third frame element 3₁ of a subsequent module via a fourth frame element 3₂ of a module. The modules in rows R1, R2, . . . , RN are connected to form the modular greenhouse 1 via a second frame element 2₂ and a first frame element 2₁ of a subsequent module.

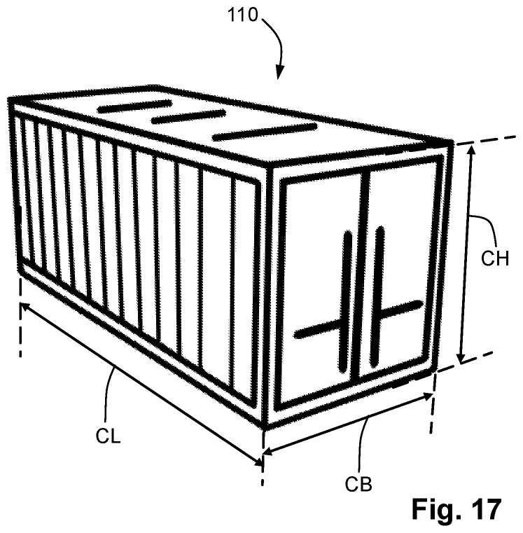

FIG. 17 shows a perspective view of a container 110 for transporting the individual components from which the individual modules 10₁, 10₂, . . . , 10_{N} of the greenhouse are constructed. The container 110 has a container length CL, a container width CB and a container height CH.

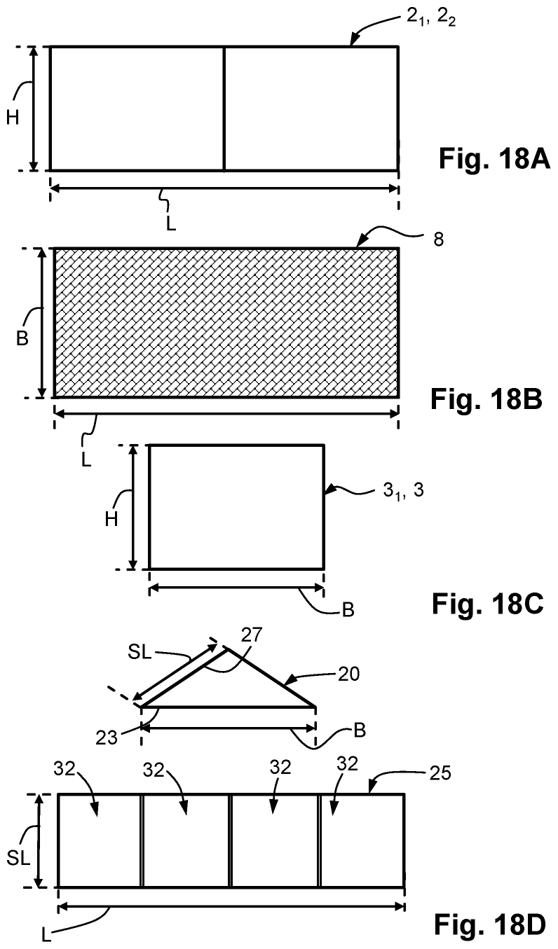

FIGS. 18A to 18D show a preferred embodiment example of the dimensioning of the individual components of each module 10₁, 10₂, . . . , 10_{N}, so that in the container 110 the available volume can be optimally used or filled with the components of each module 10₁, 10₂, . . . , 10_{N}, in order to thus save transport volume or transport costs to the installation site. Components of each module 10₁, 10₂, . . . , 10_{N} are: a first frame element 2₁, second frame element 2₂, a third frame element 3₁, a fourth frame element 3₂, at least two roof structures 20 (only one shown), at least one transparent cover 25 comprising at least one photovoltaic element 32. If necessary, another component of the modules 10₁, 10₂, . . . , 10_{N} may be a floor element 8.

The first frame element 2₁ and the second frame element 2₂ have a length L and a height H. The third frame element 3₁ and the fourth frame element 3₂ have a width B and a height H. The floor element 8 has a length L and a width B. The roof structure 20 has a base 23 with a width B and at least one leg 27 with a leg length SL. The at least two roof structures 20 of each module 10₁, 10₂, . . . , 10_{N} respectively support and hold the transparent cover 25, which has a length L and a width SL substantially equal to the leg length SL of the roof structure 20.

The individual modules 10₁, 10₂, . . . , 10_{N} can be pre-assembled in the factory and then displayed and connected to each other at the construction site for the greenhouse 1. The components of the modules 10₁, 10₂, . . . , 10_{N} can be pre-assembled for a volume-saving transport. It is advantageous if the components of the modules 10₁, 10₂, . . . , 10_{N} are transported in a container 110 so that the components of the modules 10₁, 10₂, . . . , 10_{N} are protected against damage.

With regard to saving or optimally utilizing the transport volume available in the container 110, it is advantageous if the length L of the first frame element 2₁ and the second frame element 2₂ is somewhat smaller than the container length CL. The width B of the third frame element 3₁ and the fourth frame element 3₂ is equal to half the length of the first frame element 2₁ and the second frame element 2₂. The height H of the first frame element 2₁, and second frame element 2₂ is equal to the height H of the third frame element 3₁ and the fourth frame element 3₂, the height H being smaller than the container height CH. The floor element 8 has a length L equal to the length L of the first frame element 2₁ and the second frame element 2₂. The roof structures 20 for each module 10₁, 10₂, . . . , 10_{N} have a width B of the base 23 corresponding to the width B of the third frame element 3₁ and the fourth frame element 3₂. The transparent cover 25 has a length L corresponding to the length L of the first frame element 2₁ and the second frame element 2₂, respectively. The width SL of the transparent cover 30 corresponds essentially to the leg length SL of the roof structure 20.

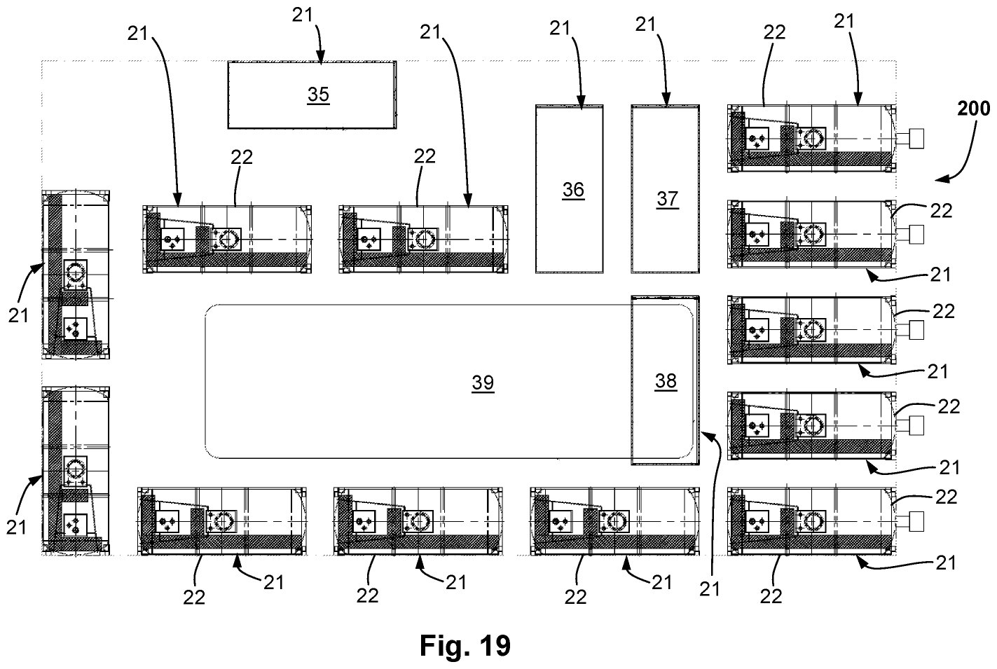

FIG. 19 shows a possible embodiment of the structure of the modular, two-stage biogas plant 200. A modular, two-stage biogas plant 200 is characterized by the fact that the modules 21 also have tanks 22 formed from two different types of tanks. Thus, the modular two-stage biogas plant 200 has as hydrolysis tanks and fermentation tanks. In the smallest configuration, the modular, two-stage biogas plant 200 has two hydrolysis tanks and one fermentation tank.

The modular, two-stage biogas plant 200 is configured by a plurality of modules 21. The modules 21 all have the same size. The same size is of particular advantage, as this greatly facilitates the transportation and production of the individual modules 21, thereby reducing costs. In addition, the equality of size of the modules 21, enables stackability or combinability. A portion of the modules 21 of the modular, two-stage biogas plant 200 is formed as tanks 22. Another module 21 of the modular, two-stage biogas plant 200 may be formed as a first enclosure 35. Similarly, another module 21 may be formed as a second enclosure 36 and still another module 21 may be formed as a third enclosure 37. The enclosures 35, 36, 37 may house elements for controlling the modular, two-stage biogas plant 200 and for extracting energy from the biogas produced by the modular biogas plant 200. Another module 21 of the modular, two-stage biogas plant 200 is a transport enclosure 38. The transport enclosure 38 may house a gas storage tank 39 for transport purposes. For operation of the modular, two-stage biogas plant 200, the flexible gas storage 39 can be rolled out of the transport enclosure 38 (housing) and thus comes to rest on an installation surface for the modular, two-stage biogas plant 200, as shown in FIG. 19.

It is self-evident for a person skilled in the art that the embodiment of the modular, two-stage biogas plant 200 shown in FIG. 19 is to be regarded merely as an example and thus does not limit the invention. It is self-evident to a person skilled in the art that the number and arrangement of the individual modules 21 can be changed depending on the scope of performance of the modular, two-stage biogas plant 200.

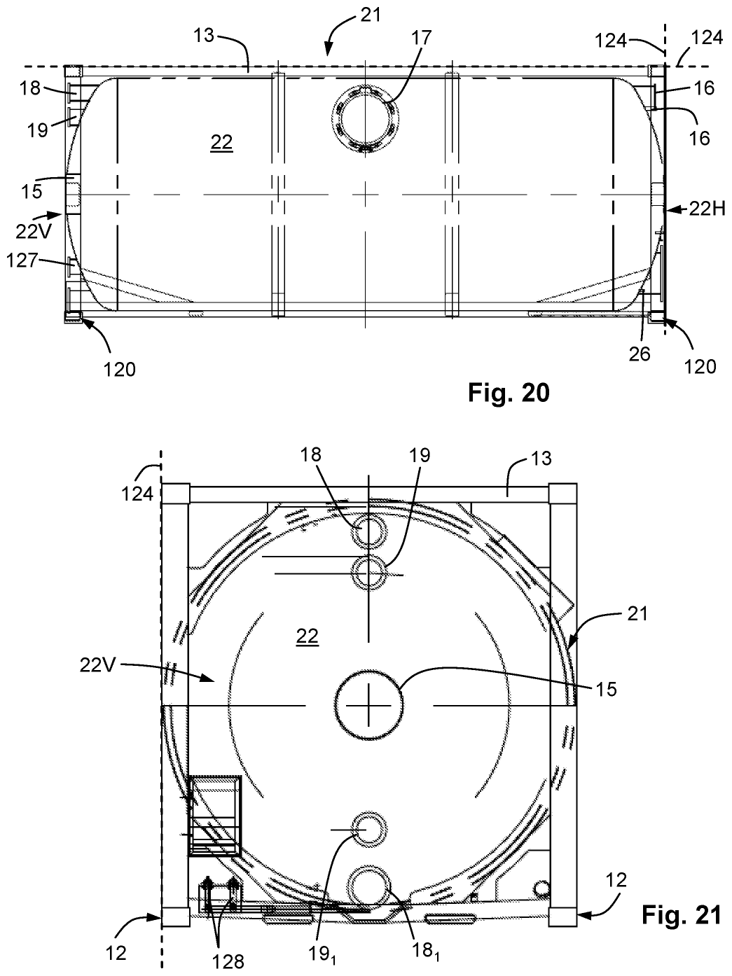

FIG. 20 shows a side view of an embodiment of a module 21 of the biogas plant 200, which is configured as a tank 22 for the modular, two-stage biogas plant 200. In the embodiment shown here, positioning elements 120 for the tank 22 are attached to a rigid frame 13. The tank 22 is surrounded by the rigid frame 13 for easy, safe and stable transportation. The frame 13 defines six side surfaces 124 that form an envelope for the tank 22. The tank 22 is disposed within the rigid frame 13 such that no connections or attachments to the tank 22 extend beyond the side surfaces 124. This has the advantage that no prefabricated connections or attachment parts of the tank 22 can be damaged during transport. The rigid frame 13 for the tanks 22 is cuboidal and has the same size as all other modules 21 of the modular, two-stage biogas plant 200.

The tank 22 has a manhole 17 attached to its side at the top of the tank 10. The position of the manhole 17 shown here is not mandatory. Depending on the requirements, the manhole 17 can be positioned as desired. It is understood that the manhole 17 is closed with a lid (not shown) during operation of the modular, two-stage biogas plant 200. At a front end 22V of the tank 22, a flanged connection 18 is provided for a gas line, a flanged connection 19 is provided for a pressure line, a flanged connection 18₁ is provided for a suction line, and a flanged connection 19₁ is provided for a gas injection. The flange connections 18, 18₁, 19 and 19₁ described herein can be provided with the appropriate lines (not shown) depending on the needs and function of the tank 22. The flange connections 18, 18₁, 19 and 19₁ are prepared so that installation can be quick and easy when setting up the modular biogas plant 200. The embodiment shown here illustrates one possible arrangement of the connections. However, the invention is not limited to the number and arrangement of the connections shown here. Furthermore, a pipe section 15 for an agitator may be provided at the front end 22V of the tank 22. If necessary, an agitator (not shown) can thus be inserted into the tank 22 at this point.

A window 16 and a level probe 14 are provided at the rear end 22H of the tank 22. The maximum filling of the tank 10 can be censored via the filling level probe 23. Furthermore, a pressure sensor 28 is still provided. The position and number of the invention of the sensor technology is only one example from many possibilities and is not to be understood as a limitation of the invention.

FIG. 21 shows a top view of the front end 22V of the tank 22. Here, too, it can be clearly seen that the side surfaces 124 of the rigid frame 13 form an envelope for the tank 22. In addition to the flanged connection 18 for the gas line, the flanged connection 19 for the pressure line, the pipe section 15 for the agitator and the flanged connection 127 for the gas injection, a heating line 128 (with flow and return) is also provided. As already mentioned in the description relating to the other drawings, the arrangement of the connections described here is merely exemplary and is not to be regarded as a limitation of the invention. The heating line 128 can thus be used to bring the interior of the tank 22, or the biomass therein, to the temperature interval required for the particular process.

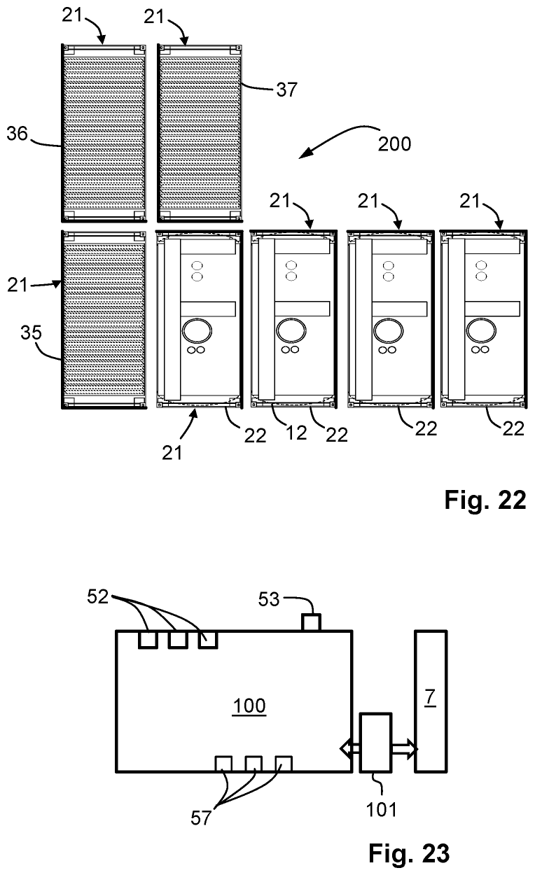

FIG. 22 shows another possible embodiment of the structure of a modular, two-stage biogas plant 200. In the embodiment shown here, the modular biogas plant 200 is composed of seven modules 21. Four of the modules 21 are formed as tanks 22. Three of the modules 21 are closed enclosures 35, 36, 37, which are in the form of standard containers (ISO sea containers with standard dimensions). It is understood that the invention is not intended to be limited to standard containers. As can also be seen from the representation of FIG. 19, the modules 21 are all of the same size. As mentioned in the description above, each of the tanks 22 is housed in a cuboidal frame 13, the frame 13 having the same size as the enclosures 35, 36 or 37. The embodiment of the modular, two-stage biogas plant 200 shown here is designed for a capacity of less than 100 kWh, but this should not be taken as a limitation of the invention.

FIG. 23 shows a schematic view of a single modular greenhouse 100. The modular greenhouse 100 provides data and parameters detected by internal sensors 52 and/or external sensors 53 of the greenhouse 100 to a local control and data acquisition unit 101 associated with the greenhouse 100. In this embodiment, the local control and data acquisition unit 101 communicates with the greenhouse 100 and the energy storage unit 7. Likewise, actuators 57 may be provided by means of which, for example in cooperation with the local control and data acquisition unit 101, the predefined and optimized conditions (temperature, humidity, light, etc.) in the modular greenhouse may be set.

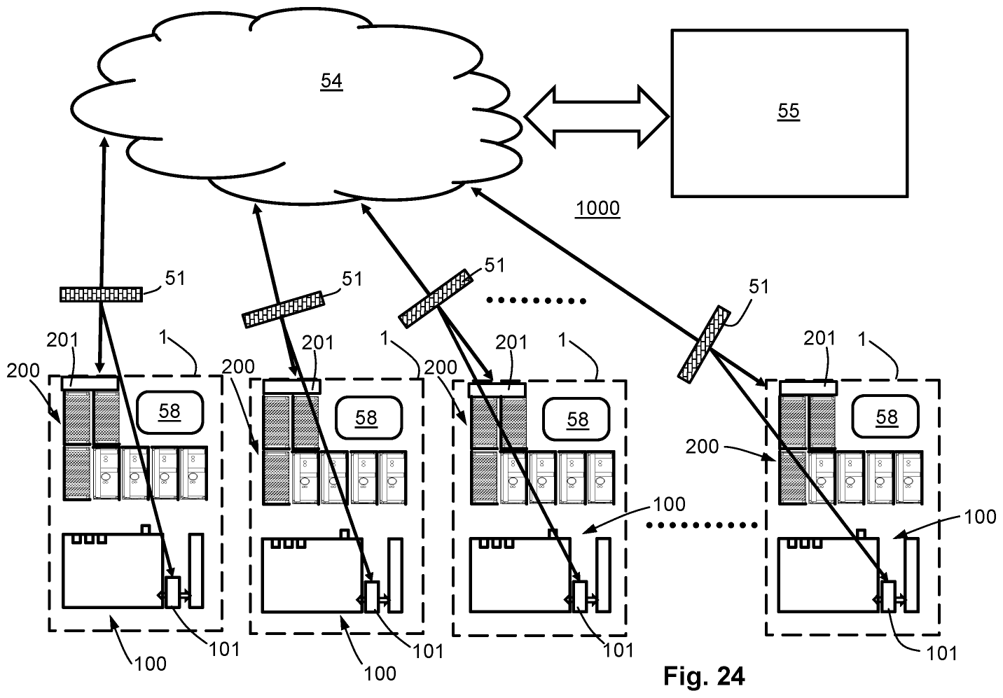

FIG. 24 shows a schematic representation system 1000 for the communication of several individual modular arrangements 1 via the cloud 54, with the central control and monitoring unit 55. Each of the arrangements 1 is defined by the modular greenhouse 100 and a modular, two-stage biogas plant 200. Data and parameters of the respective arrangement 1 are supplied via the local control and data acquisition unit 101 of the modular greenhouse 100 and the control and monitoring unit 201 of each modular, two-stage biogas plant 200. The respective arrangement 1 communicates with the cloud 54 via a firewall 51 and the Internet. The cloud 54 itself then communicates with the central control and monitoring unit 55. From the central control and monitoring unit 55, instructions, commands, messages, etc. reach the respective arrangements 1 or the local control and data acquisition units 101 of the modular greenhouses 100 and the control and monitoring units 201 of each modular, two-stage biogas plant 200 via the cloud 54, the Internet and the firewall 51. Likewise, each of the arrangements 1, may be associated with at least one user interface 58. The user interfaces 58 may receive, for example via a WLAN, the messages and/or alerts generated by the central control and monitoring unit 55. Via the user interfaces 58, these can be displayed to the operator of the local arrangement 1. The operator is thus notified centrally whether a fault occurs in the respective modular greenhouse 100 and/or the modular, two-stage biogas plant 200 of the arrangement 1, which, for example, requires a current intervention by the operator himself. Likewise, it is possible that the operator is already informed in advance about possibly upcoming repairs or replacement of components of the modular greenhouse 100 and/or the modular, two-stage biogas plant 200 of the arrangement 1. Although only identical arrangements 1 are shown in the embodiment of FIG. 24, this should not be taken as a limitation of the invention. It is self-evident to one skilled in the art that different configurations of arrangement 1 can also be centrally monitored and controlled.

The application has been described with reference to preferred embodiments. However, it is conceivable to a person skilled in the art that variations or modifications of the invention can be made without leaving the scope of protection of the claims below.

### LIST OF REFERENCE NUMERALS

1 Arrangement
2₁ First frame element
2₂ Second frame element
3₁ Third frame element
3₂ Fourth frame element
4 Roof
5 Roof surface
6 Transparent side surface
7 Energy storage unit
8 Floor element
9 Floor plan
101, 10₂, . . . 10_{N} Modul
11 Outer side
12 Longitudinal side
13 Rigid frame
14 Level probe
15 Pipe section
16 Window
17 Manhole
18 Connection
18₁ Connection
19 Connection
19₁ Connection
20 Roof structure
20₁ First leg
20₂ Second leg
21 Modul
22 Tank
2211
22V front end
23 Base
24 Support
25 Photovoltaic module
26 Top
27 Leg
28 Pressure sensor
29 Frame
30 Transparent cover
32 Thin-film solar cell
34 Recess
35 First enclosure
36 Second enclosure
37 Third enclosure
38 Transport enclosure
39 Gas storage tank
40 Lamella
42 Axe
44 Surface
45 Rectangular base
51 Firewall
52 Internal sensor
53 external sensor
54 Cloud
55 Central control and monitoring unit
57 Actuator
100 Modular greenhouse
101 Control and monitoring unit
110 Container
120 Positioning element
124 Side surface
127 Flanged connection
128 Heating line
150 Communication link
200 Biogas plant
201 Further control and monitoring unit
202 Gas storage
300 Energy
1000 System
A-A Section line
B Width
B-B Section line
CB Width
CH Height
CL Length
H Height
L Length
R1, R2, . . . , RN Row
SL Leg length
S1, S2, . . . , SN Column
X Direction

What is claimed is:

1. An arrangement for the cultivation of plants and for the utilization of biomass waste comprising:
a modular greenhouse composed of a plurality of modules;
a control and monitoring unit associated with the modular greenhouse;
a modular two-stage biogas plant consisting of several modules, wherein the modules of the modular two-stage biogas plant are partly formed as tanks, comprising at least two hydrolysis tanks and at least one fermentation tank;
a further control and monitoring unit is associated with the two-stage biogas plant; and
a communication link between the control and monitoring unit of the modular greenhouse and the further control and monitoring unit of the modular, two-stage biogas plant, so that energy from the biogas in the form of light and/or heat is used on request for the cultivation of at least plants in the modular greenhouse.

2. The arrangement according to claim 1, wherein the modules of the two-stage biogas plant all have the same size and are stackable.

3. The arrangement according to claim 1, wherein each module of the greenhouse, has a roof with at least one transparent roof surface supporting at least one photovoltaic module which is at least partially transparent or adjustable with respect to transparency, and is provided with an energy storage device.

4. The arrangement according to claim 3, wherein the energy storage device is a battery.

5. The arrangement according to claim 3, wherein the energy storage device is a power-to-gas system, with which hydrogen or methane can be generated and stored from the electricity of the photovoltaic modules, or a power-to-liquid system, with which methanol can be generated and stored from the electricity of the photovoltaic modules.

6. The arrangement according to claim 1, wherein in the hydrolysis tanks of the two-stage biogas plant the biomass is exposed to a temperature range of 40 to 65° C. and to a ph value of 2 to 9.

7. The arrangement according to claim 1, wherein in the at least one fermenter tank the biogas is produced in a second temperature range of 35 to 60° C. and at a pH value of 6.5 to 8.5.

8. The arrangement according to claim 1, wherein enclosures form another portion of the modules of the two-stage biogas plant and the enclosures house elements for controlling the modular, two-stage biogas plant and for generating energy from the biogas produced by the two-stage modular biogas plant.

9. A system of at least one arrangement for the cultivation and utilization of biomass comprising:
at least one arrangement, which is composed by a modular greenhouse and a modular two-stage biogas plant;
a plurality of modules defines the modular greenhouse and a control and monitoring unit is associated with the greenhouse;
a further control and monitoring unit is associated with the modular two-stage biogas plant, wherein the modular two-stage biogas plant consists of several modules and the modules of the modular two-stage biogas plant are partly formed as tanks, having at least two hydrolysis tanks and at least one fermentation tank;
a communication link between the control and monitoring unit of the arrangement of the modular greenhouse and the further control and monitoring unit of the modular, two-stage biogas plant; and
a central control and monitoring unit is communicatively connected to a control and data acquisition unit of the modular greenhouse and to a further control and monitoring unit of the modular, two-stage biogas plant.

10. The system according to claim 9, wherein a firewall is provided for each arrangement, wherein the control and data acquisition unit of each modular greenhouse and the further local and the control and monitoring unit of each modular two-stage biogas plant is connected via the firewall and a cloud to the central control and monitoring unit.

11. The system according to claim 9, wherein, wherein the modules of the two-stage biogas plant all have the same size and are stackable.

12. The system according to claim 9, wherein each module of the greenhouse, has a roof with at least one transparent roof surface supporting at least one photovoltaic module which is at least partially transparent or adjustable with respect to transparency, and is provided with an energy storage device.

13. The system according to claim 9, wherein in the hydrolysis tanks of the two-stage biogas plant the biomass is exposed to a temperature range of 40 to 65° C. and to a ph value of 2 to 9.

14. The system according to claim 9, wherein in the at least one fermenter tank the biogas is produced in a second temperature range of 35 to 60° C. and at a pH value of 6.5 to 8.5.

15. An arrangement for the cultivation of plants and for the utilization of biomass waste comprising:
a modular greenhouse composed of a plurality of modules, wherein each of the modules is defined by a first frame element, a second frame element, a third frame element, a fourth frame element and a roof, having at least two roof structures and holding at least one transparent roof surface;
at least one photovoltaic module is mounted on the roof surface, wherein the photovoltaic module is designed to be partially transparent or adjustable in terms of transparency;
a control and monitoring unit associated with the modular greenhouse;
a modular two-stage biogas plant consisting of several modules, wherein the modules of the modular two-stage biogas plant are partly formed as tanks, configured by at least two hydrolysis tanks and at least one fermentation tank;
a further control and monitoring unit is associated with the two-stage biogas plant;
a communication link between the control and monitoring unit of the modular greenhouse and the further control and monitoring unit of the modular, two-stage biogas plant, so that energy from the biogas in the form of light and/or heat is used on request for the cultivation of at least plants in the modular greenhouse; and
a firewall is assigned to each arrangement, wherein the control and data acquisition unit of the modular greenhouse and the further local and the control and monitoring unit of the two-stage biogas plant communicate via the firewall with the outside world.

16. The arrangement according to claim 14, wherein the hydrolysis tanks of the two-stage biogas plant the biomass is exposed to a temperature range of 40 to 65° C. and to a ph value of 2 to 9.

17. The arrangement according to claim 15, wherein in the at least one fermenter tank the biogas is produced in a second temperature range of 35 to 60° C. and at a pH value of 6.5 to 8.5.

18. The arrangement according to claim 15, wherein the photovoltaic module is defined by a frame with at least one thin-film solar cell, an energy storage device is connected to the at least one photovoltaic module of each module of the modular greenhouse, and the control and monitoring unit of the modular greenhouse is communicatively connected to internal sensors, external sensors and a plurality of actuators of the modular greenhouse, wherein a central control and monitoring unit determines setting variables for the actuators of the modular greenhouse on the basis of the data from the internal sensors and external sensors and in conjunction with predefined setpoint values of the modular greenhouse and controls the actuators accordingly.
